(19) United States
(12) Patent Application Publication
(10) Pub. No.: US 2002/0138099 A1
(43) Pub. Date: Sep. 26, 2002

(54) Method of and device for treating infections in living organism

(76) Inventors: Jury Vladimirovich Markin, Moscow (RU); Jury Konstantinovich Volikov, Moscow (RU)

Correspondence Address: John B. Hardaway, III, NEXSEN PRUET JACOBS & POLLARD, LLC, P.O. Box 10107, Greenville, SC, 29603, US

(21) Appl. No.: 09/860,708

(22) Filed: May 18, 2001

(30) Foreign Application Priority Data
Mar. 23, 2001 (RU) 2001107589

(51) Int. Cl. A61N 1/00

(52) U.S. Cl. 607/1

(57) ABSTRACT

The method of treatment proceeds in that during one session an individual information field of a living organism in which infectious agents are present is registered, this field is combined with an additional external information field which is obtained by recording, storing and reproducing the information characteristics of infectious agents. The combined information field is processed and returned into the living organism.

The device is made up of a unit for recording, storing and reproducing the information characteristics of infectious agents, connected to an apparatus for bioresonance therapy. This unit comprises connected in a definite manner: a source of primary information in a glass cylinder with a helical antenna, an amplifier, a pulsed source of noncoherent radiation, a generator, an electronic commutator coupled to a controller connected to a computer keyboard, interface unit and a liquid-crystal display, and an output amplifier connected to the electronic commutator to which a reprogrammable permanent storage is coupled.

### FIELD OF THE ART

The present invention relates to medicine and more particularly to a method of and a device for treating infections in a living organism.

The present invention may be used for combating viral infections, for harmonizing the organism functioning, for enhancing the protective forces of the organism, and also for eliminating toxins from the organism.

### BACKGROUND OF THE INVENTION

In 1977, Doctor F. Morell and Engineer E. Rasche (Germany) proposed a biophysical method—MORA-therapy, which later on received the name of “bioresonance” therapy. The founder of this method is Medtronic GMBH (Germany).

The bioresonance MORA-therapy makes it possible to exercise an integrated approach to diagnostics and therapy by using natural superfine electromagnetic oscillations of human body.

The organism and its functioning systems are sources of extremely weak electromagnetic oscillations in a broad spectrum of frequencies. Electromagnetic oscillations constitute a control level, they stimulate and control all the vital activity processes in the organism. Under the effect of pathogenic factors there originate new sources of electromagnetic oscillations, not typical of the organism. A disturbance of the dynamic equilibrium between the physiological and pathological oscillations gives rise to an information blockade which gives an impulse to triggering pathological reactions, including the formation of toxins. This process is bioenergetically treatable.

Treatment of the HIV-infection is one of major problems of modem medicine. In recent years the number of HIV-infected persons in the world has been growing steadily. At present there are no medicinal preparations which could completely save patients from the HIV-infection, though preventing its progression is quite realistic.

Today, in the case of clinical symptoms of AIDS, antiretroviral therapy is indicated to all HIV-infected patients. If the course of the disease is asymptomatic, it is more difficult to decide upon the therapy. Early therapy can prevent progressing diseases, but it is not free from definite disadvantages: deterioration of the quality of life because of undesirable effects of the antiretroviral therapy, possibility of an early development of a stable virus, and an unknown remote toxicity of antiviral medicaments.

Known in the art is a method of diagnostics and therapy of a living organism (RU 2065297, C1), which consists in recording an individual information field of a living organism, measuring, evaluating, processing, and returning it to the same organism. The individual information field of a living organism is processed with taking into account an additional external information field by combining it with the processed individual information field, and the resulting field is returned to the same or to another living organism.

The patient's electromagnetic signals are transmitted through electrodes to an apparatus, in which they are divided into physiological and pathological ones. Physiological oscillations can be amplified, and pathological oscillations are returned through the electrodes in an electronically inverted form to the patient. In this way the objective of the treatment—to attenuate the pathological signals and intensify the physiological signals—is achieved. The patient during the treatment is in a loop with the instrument, so that curative signals are constantly adjusted to changes occurring in the patient, whereby the physiological dynamic equilibrium is gradually restored and the process of restoring the regulation starts.

A device for the diagnostics and therapy of a living organism is known (RU 2065297, C1), which comprises an apparatus for bioresonance therapy, provided with electrodes. Bioresonance therapy is the treatment of man with the use of his individual electromagnetic oscillations. In this method with the help of this apparatus the entire spectrum of frequencies is affected. A particular infection cannot be destroyed, because there is no standard which could be supplied in an inverted form into the living organism or such a standard is not pure enough, does not possess clear-cut characteristics of infectious agents. In other words, it appears that there is no way for an exact comparison of the characteristics of the electric signals of infectious agents with the characteristics of infectious agents in a living organism. As a consequence, because of the absence of sufficiently complete suppression of the virus signal, there are neither sufficiently pure and reliable diagnostics nor sufficiently effective treatment.

### BRIEF DESCRIPTION OF THE INVENTION

It is an object of the present invention to provide a method of treating infections in a living organism and a device for treating infections in a living organism, which make it possible, owing to the creation of clear-cut, pure characteristics of infectious agents, to enhance the accuracy of comparing the characteristics of the electric signals of infectious agents with the characteristics of infectious agents in a living organism and, as a consequence, to enhance the effectiveness of treating the living organism.

Said object is accomplished by that in a method of treating infections in a living organism, which consists in that during one session an individual information field with the infectious agents present therein is recorded, said individual information field of the living organism with the infectious agents present therein is combined with an additional external information field, the combined information field is processed and returned into the living organism; according to the invention said additional information field being obtained by preparing, recording, subsequent storing and reproducing the information characteristics of infectious agents.

It is preferable that the duration of one session should be from 20 to 40 minutes.

It is also preferable that the seances should be carried with an interval between them of from 24 hours to several months.

The combined information field may be recorded on a material medium.

The said object of the invention is accomplished by that a device for treating infections in a living organism, comprising an apparatus for the bioresonance therapy, provided with electrodes, according to the invention, comprises a unit for recording, storing and reproducing the information characteristics of infectious agents, connected to the apparatus for the bioresonance therapy, said unit having a source of primary information, arranged in a glass cylinder around which a helical antenna is secured, connected to an amplifier, a pulsating source of non-coherent radiation, arranged with the possibility of acting on the source of primary information and connected to a generator, an electronic commutator whose input is connected to an amplifier, a reprogrammable permanent storage on n-MOS structures, connected to the electronic commutator, the electronic commutator being coupled to a controller which is connected to a computer keyboard, to an interface unit and to a liquid-crystal display, an output amplifier whose input is coupled to the output of the electronic commutator, and the output of the output amplifier being connected to the input of the apparatus for the bioresonance therapy.

The present method of and device for treating infections in a living organism make it possible, owing to the creation of clear-cut, pure characteristics of infectious agents, to enhance the accuracy of comparing the characteristics of the electric signals of infectious agents with the characteristics of infectious agents in the living organism and, as a consequence, to enhance the effectiveness of treating the living organism.

Since in the present method vanishingly weak electromagnetic signal levels are used, comparable with the levels of the electromagnetic signals of the very organism, the method has no contraindications. The present method of treating may be used for suppressing HIV replication and for substantially reducing the virusemia to a level practically safe to those around, and also for attaining by the infected person the quality of life he had before the infection.

### BRIEF DESCRIPTION OF DRAWINGS

The invention is further explained by a particular example of its embodiment and by the accompanying drawings, in which:

FIG. 1 shows a general view of a device for treating infections, according to the invention;

FIG. 2 shows a structural diagram of a unit for preparing, recording, storing and reproducing the information characteristics of infectious agents, according to the invention;

FIG. 3 shows a cluster with infection molecules before the bioresonance treatment according to the method of the invention;

FIG. 4 shows a cluster with infection molecules after the bioresonance treatment according to the method of the invention.

### DETAILED DESCRIPTION OF THE INVENTION

The method of treating infections in a living organism, according to the invention, consists in that during one session an individual information field of the living organism with infectious agents present therein is recorded, then said individual information field of the living organism with the infectious agents present therein is combined with an additional external information field. The combined information field is processed and returned into the living organism. The additional information field is obtained by preparing, recording, subsequent storing and reproducing the information characteristics of infectious agents.

The organism and its functional systems are a source of extremely weak electromagnetic oscillations. In the case of using electromagnetic fields, oscillations are sensed with the help of a system of spatially distributed pickups—electrodes which read oscillations in different areas and zones of the human organism, for instance, at biologically active points (BAP) and in biologically active zones (BAZ) on the skin.

The electrodes function as a receiving antenna which receives the patient's electromagnetic individual information field. Then this field comes along electric wires to an apparatus for bioresonance therapy. An additional electromagnetic external information field, obtained by preparing, recording, subsequent storing and reproducing the information characteristics of infectious agents, comes to the same apparatus.

For obtaining an additional external information field, a definite infection is grown in a definite concentration, then the information characteristics of the infection—of infectious agents are recorded at the maximum concentration of the infection into a unit for recording, storing and reproducing the information characteristics of infectious agents. This unit is coupled in a preset manner to the apparatus for bioresonance therapy. After both electromagnetic fields have come to the apparatus, they are processed and then fed to a transmitting antenna—to an electrode which is installed, as was indicated above, at definite BAD or in definite BAZ.

Thus, having passed special processing (spatial-temporal, frequency, nonlinear filtration, separation) in the apparatus, the oscillations from the output of the instrument are returned with the help of the same electric wires and electrodes to the patient. In the course of treatment the patient and the instrument make up a closed loop of adaptive regulation, as a result of which the processed oscillations are returned again to the patient. Therefore electromagnetic stimulation parameters are controlled by the patient's signals, and the action in this case is maximally individualized.

Carrying out one session of treatment results in destruction of the information field of infectious agents in the patient, that is, the additional external information field—the signal about a definite infection—is suppressed by the very infection signal present in the patient. The duration of one session is from 20 to 40 minutes. Sessions may be carried out every day, every other day, once a week, once a month, once in three months. The session duration and periodicity are selected for each patient individually and depend on the gravity of the disease and on the kind of infection. The combined information may be recorded on a special material medium (water, homeopathic granules, magnetic tape, etc.) with a view to using (e.g., uptaking) the obtained bioresonance preparations for subsequent therapy. For instance, if the combined information is recorded on homeopathic granules, these granules can be used later on for the out-patient treatment of a particular patient.

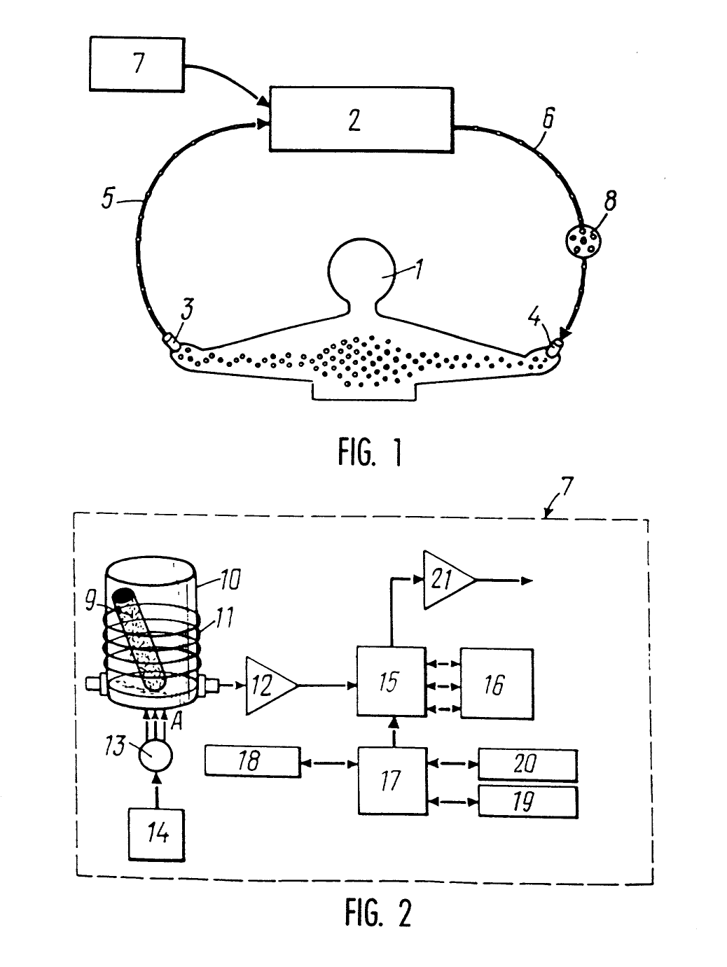

FIG. 1 shows a general view of a device for treating infections of living organism 1. The device comprises an apparatus 2 for bioresonance therapy with electrodes 3, 4. The electrodes 3, 4 are connected by wires 5, 6 to the input and output of the apparatus 2, respectively. The electrodes 3, 4 are spaced and positioned in the right and left hands of living organism 1, respectively. The device is also provided with a unit 7 for recording, storing and reproducing the information characteristics of infectious agents—definite infections—viruses, which is connected to a second input of the apparatus 2. The device also comprises a resonator 8 for recording medicaments, wherein a combined information field is recorded on a material medium. Dots in FIG. 1 represent an electromagnetic field. FIG. 2 shows a structural diagram of the unit 7 for recording, storing and reproducing the information characteristics of infectious agents. The unit 7 comprises a source 9 (FIG. 2) of primary information, arranged in a glass cylinder 10 around which a helical antenna 11 is secured, connected to an amplifier 12; a pulsating source 13 of noncoherent radiation, arranged with the possibility of acting on the source 9 of primary information and connected to a generator 14. The source 9 of primary information is a conventional sealed ampoule containing a live virus, i.e., an infection of definite kind, for instance, a herpes virus, a hepatitis virus, HIV-infection, etc. The unit 7 comprises also an electronic commutator 15 whose input is connected to the amplifier 12. The unit 7 comprises a reprogrammable permanent storage 16 on nMOS structures, connected to the electronic commutator 15. The electronic commutator is coupled also to a controller 17. The controller 17 is connected to a computer keyboard 18, to an interface unit 19 and to a liquid-crystal display 20. The input of an output amplifier 21 is coupled to the output of the electronic commutator 15. The output of the output amplifier 21 is connected to the input of the apparatus 2 (FIG. 1) for the bioresonance therapy. The direction of radiation from the source 13 of noncoherent radiation is shown in FIG. 2 by arrows A.

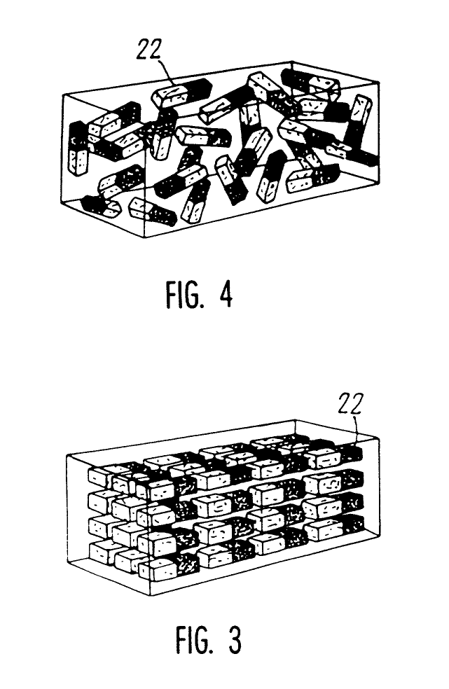

FIGS. 3, 4 show a cluster with molecules 22 of infections before and after processing thereof by the method of bioresonance treatment according to the invention, respectively. The device for treating infections in a living organism operates in the following manner. The apparatus 2 for bioresonance therapy (FIG. 1) is adjusted for operating in a standard mode, that is, for operating during 7 seconds and a subsequent break of 3 seconds (at that moment the patient's organism is at rest); a signal inversion mode is also switched-on. The patient's electromagnetic signals are transmitted through the electrodes to the apparatus 2, that is, the signal from the human organism 1 comes via the wire 5 to the first input of the apparatus 2 for bioresonance therapy; the individual information field of the living organism 1 is thus read.

The signal about infectious agents comes from the unit 7 for recording, storing and reproducing the information characteristics of infectious agents to the second input of the apparatus 2 for bioresonance therapy. These signals are amplified, the gain factor of the signal from the organism 1 being higher than of the signal about the infectious agents. Then these signals are added together and after that they are inverted and sent back through the wire 6 to the human organism 1, i.e., the processed combined information field is returned to the living organism 1.

Separate virus molecules 22 (FIG. 3) do not affect the organism uniformly. They accumulate in a definite location in the form of a cluster and are retained within it by electromagnetic linkages. When a signal about infection is fed to the cluster with a shift through 180°, the radiations of the molecules 22 are suppressed and the linkages are destroyed, so that the signal of the infectious agents becomes completely suppressed and the cluster becomes destroyed.

The operation of the unit 7 (FIG. 2) for recording, storing and reproducing the information characteristics of infectious agents will be considered in more detail hereinbelow.

The source 9 of primary information is illuminated by the pulsating source 13 of noncoherent radiation, which is controlled by the generator 14. The field from an infectious agent can be detected by human receptors at a distance of 0.5 cm.

When an infectious agent is excited by the source 13 of noncoherent radiation (red light-emitting diode with the frequency of 5 kHz), the field increases to 0.5 m. This strong field is picked up by the helical antenna 11, amplified, and comes via the electronic commutator 15 to the reprogrammable permanent storage 16. At that moment write signal is fed from the controller 17. The commutator 15 is controlled by the controller 17. The controller 17 can be controlled from the keyboard 18 or from the computer interface unit 19. The operation modes of the controller 17 are displayed on the liquid-crystal display 20.

The output signal from the reprogrammable permanent storage 16 through the commutator 15 comes to the output amplifier 21 and further to the input of the apparatus 2 for bioresonance therapy.

For storing information use is made of the reprogrammable permanent storage 16 on nMOS-structures. Such storage devices are based on the principle of the physical phenomenon of charge storage at the interface of two different dielectric media or of a conducting medium and a dielectric medium.

The metal-oxide-semiconductor gate is made “floating”, not connected with other elements of the circuit. Such a gate is charged with the avalanche injection current (whence the widespread name of LSI storage devices on structures of such type: avalanche-injection-floating-gate) of a part of the charge carriers originating when a large voltage (to 30 V) is fed to the transistor drain, causing an avalanche breakdown of the drain. As a result of such charging of the gate, the current flowing through the transistor also changes, and this is detected by the amplifier as the transistor is selected by decoding circuits. Since the transistor gate is surrounded from all sides with an insulating oxide, the charge leakage is very small, and a long-term storage of the information is ensured. To erase the information, the circuit is irradiated through a transparent window in its package with short-wave ultraviolet radiation from a luminescent lamp, which increases the leakage current in the insulating oxide and favors dissipation of the charge being stored.

Some dielectric changes occur as the electromagnetic field is fed. The capacity and attenuation changes are similar, and therefore it may be assumed that the process of conduction hopping is affected.

The magnetic field excited by the current of synchronous “hopping” of atoms (protons) between neighboring atoms brings about an electric field sufficient for the origination of self-oscillations at a frequency determined by the applied electromagnetic field at the moment of charging with the avalanche-injection current. However, this is only a classical picture of the physical phenomenon.

The most important theoretical developments made in recent years are published by Del Guidiche and his colleagues who adopted the earlier refuted theoretical postulate about the interaction in a quantized field (F. A. Wolf, “Taking the Quantum Leap”, New York, Harper and Row, 1981, pp. 65-66).

Proceeding from this theory and taking into account only the main physical constants, the coherent component of silicon must be coherent in the ground state and contain coherence domains. Each domain will contain in-phase oscillating molecules. These domains are separated by noncoherent areas.

Coherent domains of silicon must be capable of communicating with each other owing to the Josephson effect, whereby frequency-to-voltage conversion becomes possible.

Quantization of magnetic flux is the fundamental property of coherence in the magnetic field. In passive physical systems the necessary coherence and long-term ordering are attained only in the absolute temperature range. In laser and living systems coherence is attained owing to dynamic processes (I.B. Cohen: “Revolution in Science”, Cambridge, Mass., Belknap Press of Harvard University Press, 1985, pp. 427-430).

In the present device well-known instruments are used. For instance, as the microprocessor controller 17 it is possible to use a controller manufactured by Microchip Technology Inc. (1997 Technical Library Second Edition CD-ROM); as the computer interface unit 19 it is possible to use a unit manufactured by Analog Devices Inc. (1996 Designer's Reference Manual, pp. 19-13); and as the liquid-crystal display 20 a display manufactured by Holtec is used (Patent No. 84545 (R.O.C.) pending: Ser. No. 08/214,079 (USA) HT 1613).

The helical antenna 11 is made from conventional copper 1 mm-diameter wire. The number of turns of the antenna 11 depends on the height of the cylinder 10. As the source 13 of noncoherent radiation a standard light-emitting diode AL 307 is used. The generator 14 is well-known and is built around a K561LE5 microcircuit (see “Integrated Microcircuits”. A Handbook, “Radio i Svyaz” Publishers, Moscow, 1984, p.319 (in Russian)). In the present device other well-known instruments are also used. For instance, as the amplifiers 12, 21 use is made of amplifiers built around a microcircuit K14019D2B (see “Integrated Microcircuits, Operational Amplifiers”, vo1.1, “Fizikomatematicheskaya Literatura” Publishers, Moscow, 1993, pp.193, 195 (in Russian)). The electronic commutator 15 is built around a K561KTZ microcircuit (see “Integrated Microcircuits”. A Handbook, “Radio i Svyaz” Publishers, Moscow, 1984, p.329 (in Russian)).makes it possible to characterize the viral load on the organism and to judge about the degree of this load not only with respPublishers, Moscow, 1984, p. 329 (in Russian)). As the reprogrammable permanent storage 16 on n-MOS structures use is made of AT27C512R or AT2764 (see “Reference Data Sheet of the ATMEL Catalog”, Germany, pp. 3-135-3-141). As the resonator 8 for recording medicaments use is made of a resonator for recording medicaments, manufactured by PITTERLING ELECTRONIC, GMBH (see the promotional material “The Result of Eighteen-year Development”, Munich, 1994, p. 4).

As the apparatus 2 for bioresonance therapy a standard apparatus for bioresonance therapy is used (see “Present-day Advances in Bioresonance MORA-Therapy”, St.Petersburg, “AEROMET” Publishers, 1998, Part II, p. 17 (in Russian)).

The present method has passed clinical tests at the Laboratory of Infection Immunology of Academician Burdenko General Military Clinical Hospital.

The amount of the HIV proviral DNA in CD4+ cells was determined in accordance with the technology based on the polymerase chain reaction, developed at the Central Research and Technical Institute of Medical Engineering and Transfusion Medicine of the Academy of Medical and Technical Sciences.

Much attention was paid to patients infected with the human immunodeficiency virus (HIV).

The treatment of this group of patients was controlled mainly on the basis of both immunological characteristics (estimation of the cellular and humoral immunity) and by proceeding from the quantitative level of the viral ribonucleic acid (HIV RNA), as well as by using a more advanced method of determining the level of the proviral deoxyribonucleic acid (HIV DNA) in blood cells. The quantitative method of polymerase chain reaction (PCR) makes it possible to characterize the viral load on the organism and to judge about the degree of this load not only with respect to the plasma, but also directly in the genes of the CD4+ cell in the course of the treatment.

The investigation comprised 14 patients with the confirmed presence of the HIV infection and having a definite AIDS symptom complex.

Blood for the investigation was sampled before the treatment and in dynamics after each session of treatment. The cellular immunity indexes—CD3+, CD4+, CD8+, CD14+, CD25+, CD29+, CD45+ were investigated twice a year. The functional properties of lymphocytes in the leukocyte migration inhibition reaction, in the lymphocyte blast-transformation reaction, and the humoral immunity indexes—A, M, G, E immunoglobulins were evaluated before and after the treatment. The amount of HIV RNA was investigated during 2 years with a 1-3 months interval between the investigations, the number of HIV copies in the blood serum being determined with the help of AMPLICOR HIV-1 Monitor test systems manufactured by Hoffmann-La Roche, Inc. For determining the viral overburdening, vegetative-resonance testing of the preparations—HIV indicators was carried out. The geopathogenic load was tested in all the patients. The treatment was carried out with selective separation of pathological oscillations. The duration of the session of treatment according to the present method was at least 20 minutes. No drug therapy was used during the treatment.

It was established that after each session of treatment the functional activity of lymphocytes according to the leukocyte migration inhibition reaction and the lymphocyte blasttransformation reaction data lowers by 50-70% of the initial level, and the amount of A and G immunoglobulins lowers on an average by 25-30%. During the initial period of the first 2-3 months the number of HIV RNA copies in blood serum lowers by as much as 4-20 times (by 20-80% after each session). It was also noted that when the number of HIV RNA in blood serum has reached 200-1500 copies/ml, further carrying out of this treatment led to the disappearance of HIV RNA in the blood serum of 5 out of 14 patients. In the remaining 9 patients (64% of the total number), with the treatment carried out further, the amount of HIV RNA in blood serum remained on the minimum level: 200-1500 copies/ml.

The cellular immunity indexes were characterized by an increase in the number of CD4+ cells by 10-15%, in the number of CD8+ cells by 20-25%, a reduction in the number of CD45+ cells (T-helpers of the second order) by 90%, and a reduction of the immunoregular index to 0.85. Other cellular immunity indexes—CD 14+, CD16+, CD5+, CD29+ were without particular changes. It is noteworthy that patients who have undergone a course of treatment in accordance with the present method displayed a considerable lowering of the organism allergization, characterized by a reduction of the amount of T-helpers of the second order (CD45+), and also by lowering of the amount of E immunoglobulins to the level of 115-125 ME. The quantitative level of the proviral DNA, a more stable index, diminishes by 30-50% per treatment.

Therefore, it can be stated that in the process of treatment a substantial lowering of the quantitative level of viral RNA in blood serum down to 200-1500 copies/ml, i.e., a 4-20-fold lowering, was observed in 9 out of 14 patients. In the remaining 5 patients complete disappearance of the HIV RNA in blood plasma was observed. An improvement in some of the laboratory characteristics of peripheral blood in all the 14 patients should also be mentioned. It should also be noted that no aggravations in the state of health of the patients were observed either in the process of treatment or after the treatment.

The method of treating infections in a living organism will be better understood from the Examples presented hereinbelow.

### EXAMPLE NO. 1

### Male Patient R., Aged 24

Human immunodeficiency virus of type 1 (HIV-1) is confirmed by laboratory techniques in 1997. Clinical manifestations of lymphoadenopathy, influenza-like manifestations in the anamnesis. Primary viral load of 57000 HIV RNA copies/ml. HIV-1 899A 1011 strains from the collection of viruses of D.I.Ivanovskii Virology Research Institute (the Russian Academy of Medical Sciences) were used. The information field of HIV-1899A viruses was recorded into the unit 7 for recording, storing and reproducing the information characteristics of infectious agents. Then the treatment was carried out in accordance with the above-described method by using a combined information field of the HIV-1 viruses and of the patient R. viruses. Recording on homeopathic granules placed in the resonator 8 for recording medicaments was performed against the treatment background in standard mode. Sessions lasted for 1 hour, were carried out with an interval of 1-2 months, and were accompanied by laboratory determination of the number of HIV RNA in 1 ml of serum with the help of AMPLICOR HIV-1 Monitor test systems (Hoffinann-La Roche, Inc., Switzerland). Between the sessions the patient every day took homeopathic granules with the characteristics of the combined information field recorded on them. The treatment was controlled by determining the viral RNA level. This determination makes it possible to characterize the viral load on the organism and to judge about the extent of this load not only in plasma, but directly in the genes, in the CD4+ cell. Blood for treatment was sampled before the treatment and in dynamics after each session of treatment. In the process of treatment during two years the amount of the RNA copies of the HIV agent reduced to the present-day resolution limits of the HIV determination methods (less than 50 copies/ml). Clinically: lymphoadenopathy manifestations disappeared, no manifestations of opportunistic infections were detected during the entire observation period.

### EXAMPLE No. 2

### Male Patient A., Aged 30

HIV infection (HIV-1) was confirmed by the method of polymerase chain reaction (PCR) in 2000. Clinically: manifestations of lymphoadenopathy, herpes manifestations and influenza-like symptoms in the anamnesis. The viral load before the commencement of the treatment was 53800 RNA copies/ml. HIV-1899A 1011 strains from the collection of viruses of D.I.Ivanovskii Virology Research Institute (the Russian Academy of Medical Sciences) were used. The information field of HIV-1899A viruses was recorded into the unit 7 for recording, storing and reproducing the information characteristics of infectious agents. Then treatment sessions were carried out by using a combined information field of the HIV-1 viruses, reproduced from the unit 7 and of the patient A. viruses in accordance with the above-described method. The session duration was 1 hour; the periodicity of the sessions was once a week during the first three months, and then once a month. By the end of the first year of observation the viral load by the PCR method is not detected. The investigation was carried out with the help of AMPLICOR HIV-1 Monitor test systems (Hoffmann-La Roche, Inc., Switzerland).

### EXAMPLE NO. 3

### Female Patient K., Aged 28

On Oct. 18, 2000, during the visit, the patient complained of discomfort, discharge from the cervical canal. In the microbiological investigation of the discharge from the cervical canal Garderella vaginalis and Ureaplasma urealyticum were isolated in the concentration of 105-106 CFU/ml. The information characteristics of the above-named infections were recorded into the unit 7 for recording, storing and reproducing the information characteristics of infectious agents. Then with the combined information field in standard mode 6, treatments were carried out in accordance with the above-described method: on Oct. 18, 2000, on Oct. 26, 2000, on Nov. 8, 2000, on Dec. 5, 2000, on Dec. 18, 2000, and on Dec. 25, 2000. The duration of each session was 20 minutes. As a result, the constituents of the individual information field of the patient with the presence of infectious agents were destroyed. In the control investigation dated Dec. 26, 2000, the earlier isolated microorganisms (Garderella vaginalis and Ureaplasma urealyticum) were not detected.

### EXAMPLE No. 4

### Female Patient B., Aged 33

On Jan. 13, 2000, during the visit, the patient complained of burning pain in the region of the genital organs. In the investigation of the discharge from the cervical canal Candida albicans was isolated in the concentration of 109 CFU/ml. The information characteristics of the above-named infection were recorded into the unit 7 for recording, storing and reproducing the information characteristics of infectious agents. Then with the combined information field in standard mode 4 sessions were carried out in accordance with the above-described method: on Jan. 13, 2000, on Jan. 28, 2000, on Feb. 10, 2000, and on Feb. 25, 2000. The duration of each session was 20 minutes. During the session the combined information field of infectious agents and of the patient B was recorded on homeopathic granules placed in the resonator 8 for recording medicaments. The homeopathic granules were taken by the patient, between the therapeutic sessions. In the control investigation dated Feb. 25, 2000 the Candida infection was not detected.

### EXAMPLE NO. 5

### Male Patient K., Aged 21

On Jan. 11, 2000, during the visit, the patient complained of pruritus in the region of the genital organs. In the microbiological investigation of the discharge from the urethra Micoplasma genitalium was isolated in the concentration of 105 CFU/ml. The information characteristics of the above-named infection were recorded into the unit 7 for recording, storing and reproducing the information characteristics of infectious agents. Then with the combined information field in standard mode 2 treatments were carried out in accordance with the above-described method: on Jan. 11, 2000 and on Jan. 25, 2000. The duration of one session was 40 minutes. In the control investigation dated Jan. 25, 2000 the Micoplasma infection was not detected.

### EXAMPLE NO. 6

### Male Patient A., Aged 42

On Sep. 5, 2000, during the visit, the patient complained of discomfort, abdominal distention. In the microbiological investigation of the intestinal tract contents ( hemolyticus staphylococcus was isolated in the concentration of 106 CFU/ml. Dysbacteriosis was diagnosed. The information characteristics of the above-named infection were recorded into the unit 7 for recording, storing and reproducing the information characteristics of infectious agents. Then with the combined information field in standard mode, that is, with standard parameters corresponding to the operation on the apparatus 2 for bioresonance therapy, 4 treatments were carried out: on Sep. 5, 2000, on Sep. 15, 2000, on Oct. 6, 2000, and on Oct. 20, 2000. The duration of one session was 30 minutes. In the control investigation dated Oct. 20, 2000 the (hemolyticus staphylococcus infection was not detected. The patient's state of health is satisfactory: no complaints.

### EXAMPLE NO. 7

### Female Patient B., Aged 36

On Nov. 27, 2000, during the visit, the patient complained of loose stools, abdominal murmur, poor passage of flatus. In the microbiological investigation of the contents of the intestinal tract there were isolated: E. Coli in the concentration of 104 CFU/ml, Staphylococcus aureus in the concentration of 105 CFU/ml, streptococci (Streptococcus sp.) and enterococci (Enterococcus sp.) in the concentration of 104 CFU/ml. Dysbacteriosis was diagnosed. The information characteristics of the above-named infection were recorded into the unit 7 for recording, storing and reproducing the information characteristics of infectious agents. Then with the combined information field in standard mode, 3 sessions were carried out in accordance with the claimed method of treating: on Nov. 27, 2000, on Dec. 9, 2000, and on Dec. 25, 2000. The duration of one treatment was 30 minutes. In the control investigation dated Dec. 25, 2000 only individual colonies of streptococci were isolated. The patient's state of health is satisfactory: no complaints.

### EXAMPLE NO. 8

### Female Patient P., Aged 54

On May 3, 2000, during the patient's visit, dysbacteriosis was diagnosed. In the microbiological investigation of the contents of the intestinal tract Serratia marcescens, hemolytic form, was isolated in the concentration of 106 CFU/ml. The information characteristics of the above-named infection were recorded into the unit 7 for recording, storing and reproducing the information characteristics of infectious agents. Then with the combined information field in standard mode, 4 treatments were carried out in accordance with the claimed method of treating: on May 3, 2000, on May 24, 2000, and on Jun. 5, 2000. The duration of one session was 20 minutes. In the bacteriological control investigation dated Jun. 5, 2000 the microorganisms isolated earlier were not detected.

### EXAMPLE NO. 9

### Male Patient S., Aged 24

On Mar. 2, 2000, during the visit, the patient complained of dysuric phenomena, pains in the lower part of the abdomen. In the microbiological investigation of the discharge of the urethra Haemophilus sp. was isolated in the concentration of 104 CFU/ml. The information characteristics of the above-named infection were recorded into the unit 7 for recording, storing and reproducing the information characteristics of infectious agents. Then with the combined information field in standard mode, 7 sessions of treatment were carried out: on Mar. 6, 2000, on Mar. 15, 2000, on Mar. 29, 2000, on Apr. 5, 2000, on Apr. 19, 2000, on Apr. 28, 2000, and on May 10, 2000. The duration of each session was 40 minutes. In the bacteriological control investigation dated May 10, 2000 this microorganism was not isolated.

### EXAMPLE NO. 10

### Female Patient R., Aged 26

On Jan. 6, 2000, during the visit, the patient complained of burning pain during urination. In the microbiological investigation of the discharge from the cervical canal (hemolyticus Staphylococcus was isolated in the concentration of 105 CFU/ml. The information characteristics of the above-named infection were recorded into the unit 7 for recording, storing and reproducing the information characteristics of infectious agents. Then with the combined information field in standard mode, 1 treatment was carried out in accordance with the above-stated method of treating on Jan. 6, 2000. The session duration was 40 minutes. As a result, the constituents of the individual information field of the patient with the presence of infectious agents were destroyed. In the bacteriological control investigation dated May 10, 2000 this microorganism was not isolated.

With the present method of treating it is possible to treat the following infections: human immunodeficiency virus of type 1 (HIV-1), Gardnerella vaginalis and Ureaplasma urealyticum, Micoplasma genitalium, Candida albicans, (hemolyticus staphylococcus, E. Coli, Staphylococcus aureus, streptococci (Streptococcus sp.) and enterococci (Enterococcus sp.), Haemophilus sp., hemolytic form of Serratia marcescens, and others.

Hence, the present method of and the device for treating infections in a living organism make it possible, owing to the creation of clear-cut, pure characteristics of infectious agents, to enhance the accuracy of comparing the characteristics of electric signals of infectious agents with the characteristics of infectious agents in the living organism and, as a consequence, to increase the effectiveness of treating the living organism.

1. A method of treating infections in a living organism, comprising the following steps:
registering during one session an individual information field of a living organism in which infectious agents are present;
obtaining an additional external information field by preparing, recording, storing, and reproducing the information characteristics of said infectious agents;
combining said individual information field of a living organism in which infectious agents are present and the additional external information field;
processing said combined information field;
returning said combined information field to said living organism after said processing thereof.

2. A method as claimed in claim 1, wherein the duration of said one session is from 20 to 40 minutes.

3. A method as claimed in claim 1, wherein sessions are carried out with an interval between them of from one day to several months.

4. A method as claimed in claim 1, wherein said combined information field is recorded on a material medium.

5. A device for treating infections in a living organism, comprising:
an apparatus for bioresonance therapy, having a first input, a second input and an output;
a first electrode connected to said first input of said apparatus for bioresonance therapy;
a second electrode coupled to said output of said apparatus for bioresonance therapy;
said first and second electrodes arranged in the left and right hands of said living organism, respectively;
a unit for recording, storing and reproducing the information characteristics of infectious agents, having an output coupled to said second input of said apparatus for bioresonance therapy;
said unit for recording, storing and reproducing the information characteristics of infectious agents comprising:
a source of primary information, placed in a glass housing;
a helical antenna secured around said glass housing;
an amplifier having an input coupled to said helical antenna and an output;
a pulsating source of noncoherent radiation, arranged with the possibility of acting on said source of primary information and having an input;
a generator having an output coupled to said input of said pulsed source of noncoherent radiation;
an electronic commutator having a first input, a second input and an output, said first input being coupled to said output of said amplifier;
a controller, an output of said controller being connected to said second input of said electronic commutator;
a reprogrammable permanent storage on n-MOS structures, connected to said electronic commutator;
said controller being connected to a computer keyboard, interface unit, and a liquid-crystal display;
an output amplifier having an input coupled to said output of said electronic commutator, and an output, which is said output of said unit for recording, storing and reproducing the information characteristics of infectious agents.
